(19) United States
(12) Patent Application Publication
(10) Pub. No.: US 2021/0405023 A1
(43) Pub. Date: Dec. 30, 2021

(54) METHOD FOR DIAGNOSING CLOSTRIDIOIDES DIFFICILE INFECTION

(71) Applicant: THE CLEVELAND CLINIC FOUNDATION, Cleveland, OH (US)

(72) Inventors: Nabin K. Shrestha, Cleveland, OH (US); Teny M. John, Cleveland, OH (US); Raed A. Dweik, Cleveland, OH (US)

(21) Appl. No.: 17/281,324

(22) PCT Filed: Oct. 4, 2019
(86) PCT No.: PCT/US19/54638

§ 371 Date: Mar. 30, 2021

Related U.S. Application Data
(60) Provisional application No. 62/742,301, filed on Oct. 6, 2018.

(51) Int. Cl. G01N 33/497; A61B 5/00; A61B 5/08; G16H 50/20; G16B 40/00

(52) U.S. Cl. CPC G01N 33/497; A61B 5/7264; A61B 5/082; G01N 2033/4977; G16B 40/00; G01N 2033/4975; G16H 50/20

(57) ABSTRACT

A method for diagnosing a subject with Clostridioides difficile infection (GDI) is described. The method includes obtaining a breath sample from the subject, obtaining a VOC profile of the breath sample using an analytic device wherein the VOC profile comprises one or more of the VOCs detected and its corresponding quantity, inputting one or more of the VOC quantities into a machine learning model stored in a non-transitory memory and implemented by a processor, and diagnosing the subject as having or not having GDI based on the output of the machine learning model.

### RELATED APPLICATIONS

This application claims the benefit of U.S. Provisional Application No. 62/742,301, filed Oct. 6, 2018, entitled METHOD FOR DIAGNOSING CLOSTRIDIUM DIFFICILE INFECTION, the entirety of which is hereby incorporated by reference for all purposes.

### TECHNICAL FIELD

The present disclosure relates generally to methods for diagnosing Clostridioides (formerly known as Clostridium) difficile infection. More specifically, the present disclosure relates to methods for diagnosing Clostridioides difficile infection by determining the quantities of volatile organic compounds present in a breath sample obtained from a subject.

### BACKGROUND

Volatile organic compounds (VOCs) are aromatic hydrocarbon end product metabolites of physiological and patho-physiological processes. VOCs are also volatile at ambient temperature. They may be endogenous, exogenous—mostly from the environment and diet, or microbial in origin. They are present in blood, different body fluids including urine, stools, and breath during normal and disease states. VOCs are transported from different organs through blood to the lungs and subsequently exhaled via breath. VOCs can be detected in the headspace (gas space above the sample) of clinical samples, where volatile components diffuse into the gas phase, forming headspace gas. Characteristic metabolome patterns have been identified in infectious and non-infectious disease states including cancer, heart failure, kidney disease and inflammatory bowel disease. They are considered the ‘finger prints’ of underlying disease processes with unique VOC profiles seen in different conditions.

Microorganisms, constituting both normal and pathogenic flora, each with their own typical enzymatic expression, produce characteristic VOC patterns. In-vitro studies have shown differences in the microorganism-specific production of VOC's in Staphylococcus aureus and Pseudomonas aeruginosa-related ventilator associated pneumonia and among yeast cultures. These VOCs come from the organism itself, may be produced as a result of host response to the infection, or both. Advantages of VOC testing include, other than its non-invasive nature, low cost and safety of the persons working on the test.

Clostridioides difficile infection (CDI) is a cause of significant morbidity, mortality and health care expenditure. Patients with CDI have a characteristic odor to their feces that has helped healthcare workers make an “olfactory diagnosis” of CDI with moderate sensitivity and specificity. This indicates the presence of a volatile molecular signature in CDI which has led investigators to identify VOC's using gas chromatography and mass spectroscopy techniques in patients with infectious diarrhea. Discrete VOC signals in stool samples from patients with CDI compared to other infectious causes has been noted. Studies done on C. difficile stool cultures using two dimensional gas chromatography time-of-flight mass spectrometry (GC×GC-TOFMS) have identified an additional 77 volatile biomarkers—broadly classified into sulfur-containing, carbonyl-containing and others—associated with CDI.

Although there a number of commercial tests that can detect CDI, the methods all have drawbacks. For example, while PCR works well for detecting the presence of C. difficile in a patient's stool sample, it cannot differentiate between infection and colonization. The VOC expression profile in patients with C.difficile is expected to have components of VOCs generated by the microorganism as well as VOCs generated as a result of host immune response to the pathogen. Thus, new methods are needed that can diagnose infection. It has been found that examination of VOC expression profiles may be able to differentiate between patients with and without CDI, better than a test that simply looks for evidence of presence of the microorganism.

### SUMMARY

In one aspect, the present disclosure provides a method for diagnosing a subject with CDI, comprising obtaining a breath sample from the subject, obtaining a VOC profile of the breath sample using an analytic device wherein the VOC profile comprises one or more of the VOCs detected and its corresponding quantity, inputting one or more of the VOC quantities into a machine learning model stored in a non-transitory memory and implemented by a processor, and diagnosing the subject as having or not having CDI based on the output of the machine learning model.

In one instance the machine learning model is developed using a population of patients with and without CDI wherein the patients have a known CDI diagnosis.

In another instance the quantity of one or more of the following VOCs can be inputted into the machine learning model: 2-propanol, acetaldehyde, acetone, acetonitrile, acrylonitrile, benzene, carbon disulfide, dimethyl sulfide, ethanol, isoprene, pentane, 1-decene, 1-heptene, 1-nonene, 1-octene, 3-methylhexane, (E)-2-nonene, ammonia, ethane, hydrogen sulfide, triethyl amine, and trimethyl amine. In a further instance, the quantity of each of these VOCs can be inputted into the machine learning model.

In another instance, the analytical device is a selected-ion flow-tube mass spectrometer (SIFT-MS) or a gas chromatographer.

In additional instances, a diagnosis of CDI can indicate that the subject is at least 70% or 80% likely to have CDI.

Also provided herein is a method for treating a subject who has been diagnosed with CDI. The method includes obtaining a breath sample from the subject, obtaining a VOC profile of the breath sample using an analytic device wherein the VOC profile comprises one or more of the VOCs detected and its corresponding quantity, inputting one or more of the VOC quantities into a machine learning model stored in a non-transitory memory and implemented by a processor, and diagnosing the subject as having or not having CDI based on the output of the machine learning model, and administering a treatment to a subject if the subject has been diagnosed with having CDI.

In some instances, the treatment comprises administration of one or more doses of an antibiotic compound, for example, metronidazole, vancomycin, fidaxomicin, or rifaximin. In other instances, the treatment comprises non-antibiotic therapy, for example, fecal bacteriotherapy, probiotic therapy, or monoclonal antibody therapy.

### BRIEF DESCRIPTION OF THE FIGURES

The foregoing and other features of the present disclosure will become apparent to those skilled in the art to which the present disclosure relates upon reading the following description with reference to the accompanying drawings, in which:

FIG. 1 illustrates a functional block diagram of an example of a system for predicting clinical parameters related to CDI based on VOC data;

FIG. 2 illustrates a functional block diagram of a second example of a system for predicting clinical parameters relating to CDI based on VOC data.

FIG. 3 shows three plots depicting sensitivity versus specificity of VOCs in exhaled breath, stool, and plasma samples of subjects.

### DETAILED DESCRIPTION

### I. Definitions

Unless otherwise defined, all technical terms used herein have the same meaning as commonly understood by one of ordinary skill in the art to which the present disclosure pertains.

In the context of the present disclosure, the singular forms “a,” “an” and “the” can also include the plural forms, unless the context clearly indicates otherwise.

The terms “comprises” and/or “comprising,” as used herein, can specify the presence of stated features, steps, operations, elements, and/or components, but do not preclude the presence or addition of one or more other features, steps, operations, elements, components, and/or groups.

As used herein, the term “and/or” can include any and all combinations of one or more of the associated listed items.

Additionally, although the terms “first,” “second,” etc. may be used herein to describe various elements, these elements should not be limited by these terms. These terms are only used to distinguish one element from another. Thus, a “first” element discussed below could also be termed a “second” element without departing from the teachings of the present disclosure. The sequence of operations (or acts/steps) is not limited to the order presented in the claims or figures unless specifically indicated otherwise.

Also herein, where a range of numerical values is provided, it is understood that each intervening value is encompassed within the disclosure. The upper and lower limits of these smaller ranges may independently be included in the smaller ranges, and are also encompassed within the disclosure, subject to any specifically excluded limit in the stated range. Where the stated range includes one or both of the limits, ranges excluding either or both of those included limits are also included in the disclosure.

The terms “individual,” “subject,” and “patient” are used interchangeably herein irrespective of whether the subject has or is currently undergoing any form of treatment. As used herein, the term “subject” generally refers to any vertebrate, including, but not limited to a mammal.

As used herein, the term “diagnosis” can encompass determining the existence or nature of disease in a subject. As understood by those skilled in the art, a diagnosis does not indicate that it is certain that a subject has the disease, but rather that it is very likely that the subject has the disease. A diagnosis can be provided with varying levels of certainty, such as indicating that the presence of the disease is 70% likely, 85% likely, or 98% likely, for example. The term diagnosis, as used herein also encompasses determining the severity and probable outcome of disease or episode of disease or prospect of recovery, which is generally referred to as prognosis.

As used herein, the terms “treatment,” “treating,” and the like, refer to obtaining a desired pharmacologic or physiologic effect. The effect may be therapeutic in terms of a partial or complete cure for a disease or an adverse effect attributable to the disease. “Treatment,” as used herein, covers any treatment of a disease in a mammal, particularly in a human, and can include inhibiting the disease or condition, i.e., arresting its development; and relieving the disease, i.e., causing regression of the disease.

As used herein, the term “biological sample” is meant to include any biological sample from a subject where the sample is suitable for VOC analysis. Suitable biological samples for determining the level of volatile organic compound (VOC) in a subject include but are not limited to bodily fluids such as blood-related samples (e.g., whole blood, serum, plasma, and other blood-derived samples), stools, and the like. Another example of a biological sample is an exhaled breath sample. A biological sample may be fresh or stored. Biological samples may be or have been stored or banked under suitable tissue storage conditions. Preferably, biological samples are either chilled after collection if they are being stored to prevent deterioration of the sample.

As used herein, the term “C. difficile colonization” refers to the detection of the C. difficile organism or its toxin in a subject in the absence of CDI symptoms.

### II. Overview

The present disclosure relates generally to a method for diagnosing a subject with CDI based on the quantity of one or more VOCs present in the subject's breath. From this diagnoses, methods of treatment are also provided.

The present disclosure is based, at least in part, on the surprising finding that there are quantifiable differences in VOCs in the breath of patients with and without CDI that allow for identification of CDI using breath analysis. The VOCs that may be used to differentiate between subjects with and without CDI can include 2-propanol, acetaldehyde, acetone, acetonitrile, acrylonitrile, benzene, carbon disulfide, dimethyl sulfide, ethanol, isoprene, pentane, 1-decene, 1-heptene, 1-nonene, 1-octene, 3-methyl hexane, (E)-2-nonene, ammonia, ethane, hydrogen sulfide, triethyl amine, and trimethyl amine among others. A machine learning model can be generated using the quantifiable differences in VOCs in in the breath of patients with and without CDI, and the output of the machine learning model can be used to diagnose a subject with CDI. This method of diagnosing CDI allows for medical professionals to carry out rapid, point-of-care tests using a clean sampling method.

In one aspect, the present disclosure provides a method of diagnosing a subject with CDI where the method first includes obtaining a breath sample from the subject. A breath sample may be collected in a container. For example, a bag such as a Mylar balloon bag can be used as a container for the breath sample. In certain instances, the ambient air that is inhaled prior to collection of a subsequent breath sample can be optionally filtered. The filter can be used to prevent viral and bacterial exposure to the subject and to eliminate exogenous volatile organic compounds from the inhaled air.

In one example, a breath sample can be collected from a subject using a collection device that includes a mouthpiece, one or more filters, and a collection bag. The breath sample can be collected using the following process: (i) the subject can carry out a tidal volume exhalation to clear residual air from the anatomic dead space; (ii) the subject can take a deep breath through a disposable micro filtered mouthpiece which can prevent exposure to viral and bacterial pathogens in the ambient air and eliminate exogenous VOCs; and (iii) the subject can carry out tidal volume exhalation back through the mouthpiece. The exhaled breath can be collected in a Mylar balloon bag.

Once a breath sample has been obtained, an analytic device can be used to generate a VOC profile of the breath sample.

With recent advances in technology, it is possible to identify thousands of substances in the breath, such as volatile compounds and elemental gases. A number of methods and analytic devices known in the art can be used to detect the presence of the VOCs in a biological sample. Exemplary methods include gas chromatography (GC); spectrometry, for example mass spectrometry, and colorimetry. A number of different forms of mass spectrometry can be used including selected-ion flow-tube mass spectrometry (SIFT-MS), quadrapole, time of flight, tandem mass spectrometry, ion cyclotron resonance, and/or sector (magnetic and/or electrostatic) mass spectrometry. For example, SIFT-MS can identify trace gases in the human breath in the parts per billion, and even the parts per trillion range.

Other spectrometry methods that may be used include field asymmetric ion mobility spectrometry and differential mobility spectrometry (DMS). DMS has several features that make it an excellent platform for VOC analysis: it is quantitative, selective, and exquisitely sensitive, with a volatile detection limit in the parts-per-trillion range.

The analytic device can be a portable or a stationary device.

In some aspects, the analytic device includes a gas collection component for receiving a breath sample. For example, the analytic device can be a mass spectrometry device with a Mylar collection bag attached directly to it.

The VOC profile generated from the analytic device may comprise one or more of the VOCs detected and its corresponding quantity. In one example, the VOC profile can include one or more of the following VOCs and its corresponding quantity: 2-propanol, acetaldehyde, acetone, acetonitrile, acrylonitrile, benzene, carbon disulfide, dimethyl sulfide, ethanol, isoprene, pentane, 1-decene, 1-heptene, 1-nonene, 1-octene, 3-methyl hexane, (E)-2-nonene, ammonia, ethane, hydrogen sulfide, triethyl amine, and trimethyl amine. In another example, the VOC profile may include all of these VOCs and their corresponding quantities. One skilled in the art would understand that an analytic device may be able to detect hundreds of VOCs in a subject's breath. The VOC profile can contain each of the detected VOCs and their corresponding quantities or a subset thereof. In one example, the VOC profile can include those where the difference in the VOC quantities between the subjects with and without CDI is significant.

Following generation of the VOC profile, one or more of the detected VOC quantities can be inputted into a machine learning model. The machine learning model can diagnose a subject with CDI. More specifically, the machine learning model can provide the likelihood that the subject has CDI. In certain instances, a diagnosis of CDI can indicate that the subject is at least 70%, 80%, or 90% likely to have CDI.

A number of machine learning models can be generated to predict whether or not a subject has CDI.

Machine Learning Models

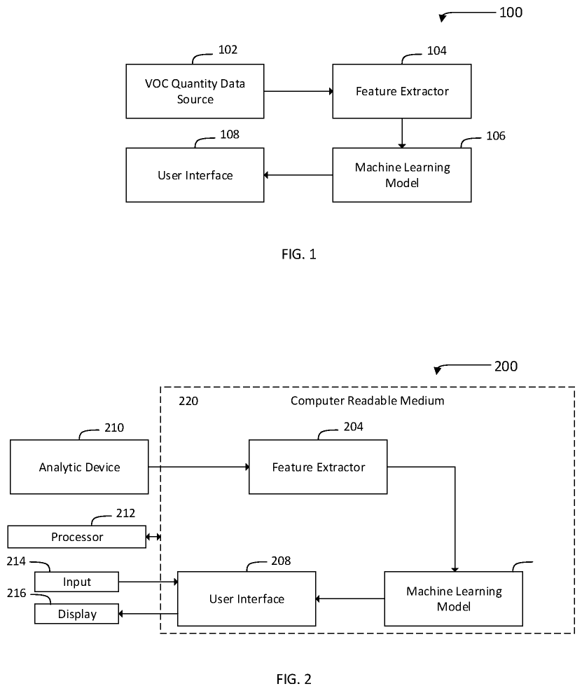

One aspect of the present disclosure is shown in FIG. 1. FIG. 1 illustrates a functional block diagram of an example of a system 100 for predicting whether or not a subject has CDI based on the VOC profile of the subject's breath. The system 100 can be implemented on one or more physical devices (e.g., servers) that may reside in a cloud computing environment or on a computer, such as a laptop computer, a desktop computer, a tablet computer, a workstation, or the like. In the present example, although the components 102, 104, 106, and 108 of the system 100 are illustrated as being implemented on the same system, in other examples, the different components could be distributed across different systems and communicate, for example, over a network, including a wireless network, a wired network, or a combination thereof. The system 100 includes a VOC quantity data source 102 that can be accessed to provide one or more VOC quantities. The VOC quantity data source 102 can include, for example, the analytic device used to generate the VOC profile and determine the quantity of one or more VOCs. The VOC quantity data source may also contain a storage medium accessible by a local bus or a network connection, or a user interface at which a user can enter information from a previously obtained VOC profile.

A feature extractor 104 generates a feature vector representing the subject from the VOC profile. For example, the feature extractor 104 can utilize the absolute or normalized quantity or concentration of one or more of the detected VOCs or one or more values derived from the VOC quantities. It will be appreciated that the feature extractor 104 can also utilize additional parameters, for example, general biometric parameters of the subject such as age and sex, and other medical diagnoses such as heart failure. These parameters can be provided, for example, from an electronic health records database via a network interface (not shown) or via a user interface 106. A machine learning model 106 determines at least one clinical parameter for the subject from the metric. It will be appreciated that the clinical parameter can represent, for example, the probability that the subject has CDI or the probability that the subject will respond to treatment for CDI. The clinical parameter provided by the machine learning model 106 can be stored on a non-transitory computer readable medium associated with the system and/or provided to a user at a display via the user interface 108.

FIG. 2 illustrates a functional block diagram of an example of a system 200 for predicting clinical parameters related to CDI. To this end, the system 200 incorporates a machine learning model 206 that generates a clinical parameter representing, for example, a CDI diagnosis or the probability that a subject will respond to treatment for CDI. In the illustrated implementation, an analytic device 210 provides VOC data, for example, the quantity of one or more VOCs detected, to a data analysis component implemented as a general purpose processor 212 operatively connected to a non-transitory computer readable medium 220 storing machine executable instructions. An input device 214, such as a mouse or a keyboard, is provided to allow a user to interact with the system, and a display 216 is provided to display VOC data and calculated parameters to the user.

The machine learning model 206 can utilize one or more pattern recognition algorithms, implemented, for example, as classification and regression models, each of which analyze the extracted feature vector to assign a clinical parameter to the user. It will be appreciated that the clinical parameter can be categorical or continuous. For example, a categorical parameter can represent the presence or absence of CDI, expected efficacy of the treatment, or binned ranges of likelihood of these categories. A continuous parameter can represent, for example, a likelihood that the subject has CDI or a likelihood that the subject will respond to treatment.

Where multiple classification and regression models are used, the machine learning model 206 can include an arbitration element can be utilized to provide a coherent result from the various algorithms. Depending on the outputs of the various models, the arbitration element can simply select a class from a model having a highest confidence, select a plurality of classes from all models meeting a threshold confidence, select a class via a voting process among the models, or assign a numerical parameter based on the outputs of the multiple models. Alternatively, the arbitration element can itself be implemented as a classification model that receives the outputs of the other models as features and generates one or more output classes for the patient.

The classification can also be performed across multiple stages. In one example, the biometric or clinical parameters for the subject can be used with a first stage of the machine learning model to generate an a priori probability that the subject has CDI. The VOC quantities for the subject can then be determined and used at a second stage of the machine learning model to generate a classification for the subject as having CDI or not having CDI. A known performance of the second stage of the machine learning model, for example, defined as values for the specificity and sensitivity of the model, can be used to update the a priori probability given the output of the second stage.

The machine learning model 206, as well as any constituent models, can be trained on training data representing the various classes of interest. The training process of the machine learning model 206 will vary with its implementation, but training generally involves a statistical aggregation of training data into one or more parameters associated with the output classes. Any of a variety of techniques can be utilized for the models, including support vector machines (SVM), regression models, self-organized maps, k-nearest neighbor (KNN) classification or regression, fuzzy logic systems, data fusion processes, boosting and bagging methods, rule-based systems, or artificial neural networks (ANN).

For example, an SVM classifier can utilize a plurality of functions, referred to as hyperplanes, to conceptually divide boundaries in the N-dimensional feature space, where each of the N dimensions represents one associated feature of the feature vector. The boundaries define a range of feature values associated with each class. Accordingly, an output class and an associated confidence value can be determined for a given input feature vector according to its position in feature space relative to the boundaries. An SVM classifier utilizes a user-specified kernel function to organize training data within a defined feature space. In the most basic implementation, the kernel function can be a radial basis function, although the systems and methods described herein can utilize any of a number of linear or non-linear kernel functions.

An ANN classifier comprises a plurality of nodes having a plurality of interconnections. The values from the feature vector are provided to a plurality of input nodes. The input nodes each provide these input values to layers of one or more intermediate nodes. A given intermediate node receives one or more output values from previous nodes. The received values are weighted according to a series of weights established during the training of the classifier. An intermediate node translates its received values into a single output according to a transfer function at the node. For example, the intermediate node can sum the received values and subject the sum to a binary step function. A final layer of nodes provides the confidence values for the output classes of the ANN, with each node having an associated value representing a confidence for one of the associated output classes of the classifier.

A k-nearest neighbor model populates a feature space with labelled training samples, represented as feature vectors in the feature space. In a classifier model, the training samples are labelled with their associated class, and in a regression model, the training samples are labelled with a value for the dependent variable in the regression. When a new feature vector is provided, a distance metric between the new feature vector and at least a subset of the feature vectors representing the labelled training samples is generated. The labelled training samples are then ranked according to the distance of their feature vectors from the new feature vector, and a number, k, of training samples having the smallest distance from the new feature vector are selected as the nearest neighbors to the new feature vector.

In the classifier model, the class represented by the most labelled training samples in the k nearest neighbors is selected as the class for the new feature vector. In a regression model, the dependent variable for the new feature vector can be assigned as the average of the dependent variables for the k nearest neighbors. It will be appreciated that k is a metaparameter of the model that is selected according to the specific implementation. The distance metric used to select the nearest neighbors can include a Euclidean distance, a Manhattan distance, or a Mahalanobis distance.

A regression model applies a set of weights to various functions of the extracted features, most commonly linear functions, to provide a continuous result. In general, regression features can be categorical, represented, for example, as zero or one, or continuous. In a logistic regression, the output of the model represents the log odds that the source of the extracted features is a member of a given class. In a binary classification task, these log odds can be used directly as a confidence value for class membership or converted via the logistic function to a probability of class membership given the extracted features.

A rule-based classifier applies a set of logical rules to the extracted features to select an output class. Generally, the rules are applied in order, with the logical result at each step influencing the analysis at later steps. The specific rules and their sequence can be determined from any or all of training data, analogical reasoning from previous cases, or existing domain knowledge. One example of a rule-based classifier is a decision tree algorithm, in which the values of features in a feature set are compared to corresponding threshold in a hierarchical tree structure to select a class for the feature vector. A random forest classifier is a modification of the decision tree algorithm using a bootstrap aggregating, or “bagging” approach. In this approach, multiple decision trees are trained on random samples of the training set, and an average (e.g., mean, median, or mode) result across the plurality of decision trees is returned. For a classification task, the result from each tree would be categorical, and thus a modal outcome can be used, but a continuous parameter can be computed according to a number of decision trees that select a given task. Regardless of the specific model employed, the clinical parameter generated at the machine learning model 206 can be provided to a user at the display 216 via a user interface 208 or stored on the non-transitory computer readable medium 220, for example, in an electronic medical record associated with the patient.

In one aspect, the machine learning model is generated using breath VOC profiles of subjects with and without CDI where the subject's CDI diagnosis is already known. In one example, the machine learning model is generated using a k-nearest neighbor classification algorithm. In one instance, the breath VOC profiles used to generate the machine learning model can include quantities of one or more of the following VOCs: 2-propanol, acetaldehyde, acetone, acetonitrile, acrylonitrile, benzene, carbon disulfide, dimethyl sulfide, ethanol, isoprene, pentane, 1-decene, 1-heptene, 1-nonene, 1-octene, 3-methyl hexane, (E)-2-nonene, ammonia, ethane, hydrogen sulfide, triethyl amine, and trimethyl amine. In some instances, the quantities of all of the aforementioned VOCs are used to generate the machine learning model. In further instances, the VOC quantities used to generate the machine learning model can include those where the difference in the VOC quantities between the subjects with and without CDI is significant.

Also provided herein are methods for treating a subject who has been diagnosed with CDI. In one aspect, the method includes obtaining a breath sample from the subject, obtaining a VOC profile of the breath sample using an analytic device wherein the VOC profile comprises one or more of the VOCs detected and its corresponding quantity, inputting one or more of the VOC quantities into a machine learning model stored in a non-transitory memory and implemented by a processor, and diagnosing the subject as having or not having CDI based on the output of the machine learning model. If the subject is diagnosed with CDI, the subject can be treated accordingly. In one instance, the treatment can comprise administration of one or more doses of an antibiotic compound. For example, metronidazole, vancomycin, fidaxomicin, or rifaximin may be administered to the patient. In other instances, the treatment can comprise non-antibiotic therapy, for example, fecal bacteriotherapy, probiotic therapy, or monoclonal antibody therapy. Colectomy can be considered for severely ill subjects.

In some embodiments, the methods include performing an additional diagnostic test for CDI. A number of such tests are known in the art and include stool tests for C. difficile toxins or toxigenic C. difficile. The stool tests include enzyme immunoassays that may or may not include lateral flow devices, and PCR.

Additionally provided herein are methods of determining if a subject is at risk for developing CDI. In one aspect, the method includes obtaining a breath sample from the subject, obtaining a VOC profile of the breath sample using an analytic device wherein the VOC profile comprises one or more of the VOCs detected and its corresponding quantity, inputting one or more of the VOC quantities into a machine learning model stored in a non-transitory memory and implemented by a processor, and determining if the subject is at risk for developing CDI based on the output of the machine learning model.

Another aspect of the disclosure is directed to diagnosing C. difficile colonization in a subject. The method can include obtaining a breath sample from the subject, obtaining a VOC profile of the breath sample using an analytic device wherein the VOC profile comprises one or more of the VOCs detected and its corresponding quantity, inputting one or more of the VOC quantities into a machine learning model stored in a non-transitory memory and implemented by a processor, and diagnosing the subject as having or not having C. difficile colonization based on the output of the machine learning model.

Additionally, one skilled in the art would understand that a medical professional can make treatment decisions based on a diagnosis of CDI or C. difficile colonization in a subject. For example, the medical professional can decide if a subject should be placed in contact isolation. Additionally, a medical professional can decide if an antibiotic should be administered to the subject.

### EXPERIMENTAL

The following example is for the purpose of illustration only is not intended to limit the scope of the appended claims.

### Example 1

The presence of volatile organic compounds (VOCs) accounts for the characteristic odor of stool in Clostridioides difficile infection (CDI). Gas chromatographic methods have identified specific signature molecules in the stool of patients with CDI. This study using selected ion flow tube mass spectrometry (SIFT-MS) shows that VOC pattern in breath had good sensitivity (87%) and moderate specificity (77%) for identifying patients with CDI.

Volatile organic compounds (VOCs) are aromatic hydrocarbon end product metabolites of physiological and pathophysiological processes. (Yoshida et al. 2012) VOCs are transported through the blood from different organs to the lungs and subsequently exhaled. Patients with Clostridioides difficile infection (CDI) have been noted to have a characteristic odor to their feces due to the presence of VOCs. (Burdette and Bernstein 2007) Previous studies using gas chromatography-mass spectrometry (GC-MS) characterized this ‘volatile molecular signature’ in the stool of patients with CDI (Rees et al. 2016). Selected ion flow tube mass spectroscopy (SIFT-MS) enables the measurement of lower concentrations (parts per billion) of VOCs in clinical samples. (Rieder et al. 2016) SIFT-MS technology has shown high discriminatory capacity in other syndromes like inflammatory bowel disease, nonalcoholic fatty liver disease, and pulmonary artery hypertension. (Kurada et al. 2015)(Alkhouri et al. 2014)(Cikach et al. 2014) The purpose of this study was to determine if VOCs in stool, blood, and breath of patients with CDI, as measured by SIFT-MS, differed from those in age-matched controls without CDI.

Methods

The cross-sectional study enrolled patients >18 years old with diarrhea who had stool tested for Clostridioides difficile by PCR. Patients with >3 episodes of diarrhea in the preceding 24 hours and an illness suggestive of C.difficile (abdominal pain, fever, elevated WBC count) with a stool specimen positive for C. difficile by PCR were considered to have CDI. The single best age and gender-matched patient, with liquid stools but negative C. difficile PCR on the same day, was selected as a control for each patient included in this study. Patients without a clinical illness compatible with CDI, patients who refused or were unable to give informed consent (e.g., due to intubation, encephalopathy, delirium, or pharmacologic sedation), patients tested during the weekends, and patients with CDI in the previous four weeks were excluded. Consecutive cases and controls identified during working days (Monday through Friday) were studied.

Stool samples sent to the microbiology laboratory for C.difficile PCR testing, and plasma samples drawn within 24 hours of stool collection were identified, and 100 μL of residual specimen were obtained.

Breath samples were collected at the patients' bedside within 24 hours of collection of the stool specimen. Initially, tidal volume exhalation was done to clear residual air from the anatomic dead space, followed by a deep breath through a disposable micro-filtered mouthpiece, which prevented exposure to viral and bacterial pathogens in ambient air and eliminated exogenous VOCs, followed by tidal volume exhalation back through the mouthpiece. We collected exhaled breath in a Mylar balloon bag.

All samples (stool, plasma, and breath) were incubated at 37° C. for 30 minutes before analysis. For stool and plasma samples, 20 mL of headspace gas was removed from the vials using a glass syringe. For breath samples, the Mylar bag was connected to the mass spectrometry device directly.

The gas from samples was analyzed using a VOICE200 SIFT-MS instrument (Syft Technologies Ltd, Christchurch, New Zealand). We obtained mass scans of the product ions generated in the chemical ionization mass spectrum from each reagent ion (H₃O+, NO+, and O₂+) were obtained in the mass scanning (MS) mode. Mass scanning between 14 and 200 atomic mass units was used to identify significant peaks at product ion masses representing unknown volatiles relating to CDI. The laboratory personnel analyzing the samples, and the microbiology technicians' aliquoting the stool and plasma samples, were blinded to the stool C. difficile test results.

VOC analysis findings were analyzed using the K-nearest neighbors (KNN) method. Model accuracy was evaluated by k-fold cross-validation with five folds. Sensitivity and specificity were determined, and receiver-operating characteristic curves generated for each sample type.

Results

Of 67 patients with positive stool C.difficile PCR screened for inclusion in the study, 36 were excluded (10 did not have a clinical illness compatible with CDI, 9 were delirious, 7 were intubated, or on high flow O₂, 4 were discharged before getting a breath sample, and 1 patient did not have a plasma sample available for testing). Each of the 31 patients that met our inclusion criteria had a matched control. The CDI and non-CDI groups were comparable with respect to mean age, race, gender, and smoking status. (Table 1).

TABLE 1
Baseline demographic characteristics

| | Cases | | Controls | | p- |
|---|---|---|---|---|---|
| Characteristic | (n = 31) | | (n = 31) | | value |
| Age (years) | 56.9 ± 15.1 | | 52.8 ± 15.3 | | 0.29 |
| Race | | | | | 0.71 |
| Caucasian | 26 | (84%) | 27 | (87%) | |
| African American | 5 | (16%) | 3 | (10%) | |
| Others | 0 | (0%) | 1 | (3%) | |
| Male | 15 | (48%) | 15 | (48%) | 0.99 |
| Body Mass Index (kg/m²) | 28.8 ± 9.6 | | 29.0 ± 7.2 | | 0.94 |
| Comorbidities | | | | | |
| Diabetes Mellitus | 9 | (29%) | 7 | (23%) | 0.56 |
| Coronary Artery Disease | 6 | (19%) | 2 | (6%) | 0.26 |
| Heart Failure | 6 | (19%) | 0 | (0%) | 0.02 |
| Chronic Obstructive | 2 | (6%) | 1 | (3%) | 0.99 |
| Pulmonary Disease | | | | | |
| Chronic Kidney Disease | 4 | (13%) | 5 | (16%) | 0.99 |
| End Stage Renal Disease | e | | NA | | — |
| Chronic Liver Disease | 1 | (3%) | 3 | (10%) | 0.61 |
| Inflammatory Bowel | 2 | (6%) | 7 | (23%) | 0.15 |
| Disease | | | | | |
| Malignancy | 12 | (39%) | 5 | (16%) | 0.05 |
| History of Transplant | 5 | (16%) | 9 | (29%) | 0.22 |
| Solid Organ Transplant | 4 | | 5 | | |
| Hematopoietic Stem Cell | 1 | | 3 | | |
| Transplant | | | | | |
| Concurrent Infection(s) | 11 | (35%) | 9 | (29%) | 0.59 |
| Smoking | | | | | 0.99 |
| Current Smoker | 5 | (16%) | 5 | (16%) | |
| Ex-Smoker | 9 | (29%) | 9 | (29%) | |
| Non-Smoker | 17 | (55%) | 17 | (55%) | |
| Alcoholism | 10 | (32%) | 10 | (32%) | 0.99 |
| Clostridioides difficile | | | | | |
| Severity | | | | | |
| Non- Severe | 18 | (58%) | NA | | — |
| Severe | 8 | (26%) | NA | | — |
| Fulminant | 5 | (16%) | NA | | — |
| Prior history of | 4 | (13%) | 1 | (3%) | 0.35 |
| Clostridioides difficile | | | | | |
| (>4 weeks ago) | | | | | |
| Peak WBC Count | 11.5 | (5.2, 20.6) | 10.1 | (5.3, 12.9) | 0.35 |
| (×109/L) | | | | | |
| Peak Serum Creatinine | 1.1 | (0.8, 4.0) | 0.9 | (0.8, 1.4) | 0.10 |
| (mg/dL) | | | | | |
| Nadir Serum Albumin | 2.9 | (2.4, 3.6) | 2.8 | (2.3, 3.3) | 0.49 |
| (g/dL) | | | | | |

Descriptive statistics reported as either mean ± standard deviation, median (Q₁, Q₃) or count (%)
Means are compared with t-tests, medians compared with Wilcoxon rank-sum tests and proportions compared with chi-square or Fisher's exact test as appropriate.

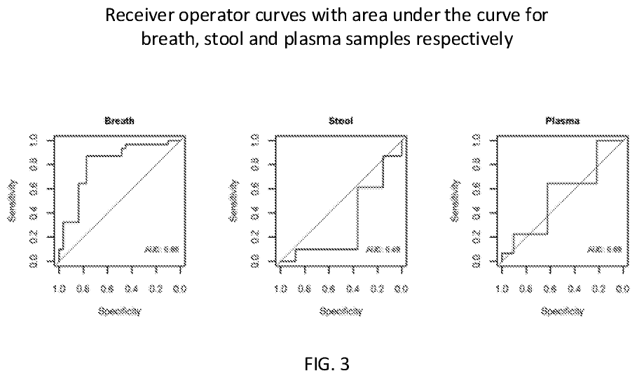

The optimal KNN classifier model was achieved with k=7, 5, and 9, for breath, plasma, and stool samples, respectively. The sensitivity/specificity for detection of CDI were 87%/77%, 67%/64%, and 61%/37%, for breath, stool, and plasma samples, respectively. Receiver-operating characteristic curves (ROC) for identification of CDI using breath, plasma, and stool samples, are shown in FIG. 3.

Model accuracy was not improved when positives were limited to those with C. difficile PCR cycle threshold (CT)<30 cycles.

Prior studies using SIFT-MS technology have demonstrated the utility of breath analysis in a wide range of non-infectious conditions, including inflammatory bowel disease, non-alcoholic fatty liver disease, and fibrosis associated with chronic liver disease. (Kurada et al. 2015). This proof-of-concept study showed that VOC patterns in breath are different in patients with and without CDI, and analysis of these differences has reasonable sensitivity and specificity to make a diagnosis of CDI.

In this study, plasma, and stool VOCs did not differentiate patients with CDI from patients with non-CDI. Prior VOC studies in C. difficile performed on direct stool samples or stool cultures showed characteristic VOC patterns attributable to C. difficile. (Garner et al. 2007)(Rees et al. 2016). In the study using direct stool samples, special care was taken to prevent the loss of VOCs including the collection in individual vials followed by sealing and freezing at −20 c within 1 hour of the passage of stool. In this study, a delay in the time of collection to the time of analysis of stool and plasma samples may have resulted in the loss of discriminant VOCs, thereby rendering the analysis inadequate to diagnose CDI.

The complete disclosure of all patents, patent applications, and publications, and electronically available material cited herein are incorporated by reference. The foregoing detailed description and examples have been given for clarity of understanding only. No unnecessary limitations are to be understood therefrom. Although the invention has been described with reference to several specific aspects, the invention is not limited to the exact details shown and described, for variations obvious to one skilled in the art will be included. The description is not meant to be construed in a limited sense. Various modifications of the disclosed aspects, as well as alternative aspects of the inventions will become apparent to persons skilled in the art upon the reference to the description of the invention. It is, therefore, contemplated that the appended claims will cover such modifications that fall within the scope of the invention.

What is claimed is:

1. A method of for diagnosing a subject with a Clostridioides difficile infection (CDI), comprising:
obtaining a breath sample from the subject;
obtaining a VOC profile of the breath sample using an analytic device wherein the VOC profile comprises one or more of the VOCs detected and its corresponding quantity;
inputting one or more of the VOC quantities into a machine learning model stored in a non-transitory memory and implemented by a processor; and
diagnosing the subject as having or not having CDI based on the output of the machine learning model.

2. The method of claim 1, wherein the machine learning model is developed using a population of patients with and without CDI.

3. The method of claim 1, wherein the analytic device is a selected-ion flow-tube mass spectrometry (SIFT-MS).

4. The method of claim 1, wherein the analytic device is a gas chromatograph.

5. The method of claim 1, wherein the one or more VOC quantities inputted into the machine learning model are selected from 2-propanol, acetaldehyde, acetone, acetonitrile, acrylonitrile, benzene, carbon disulfide, dimethyl sulfide, ethanol, isoprene, pentane, 1-decene, 1-heptene, 1-nonene, 1-octene, 3-methylhexane, (E)-2-nonene, ammonia, ethane, hydrogen sulfide, triethyl amine, and trimethyl amine.

6. The method of claim 1, wherein the VOC quantities inputted into the machine learning model comprise 2-propanol, acetaldehyde, acetone, acetonitrile, acrylonitrile, benzene, carbon disulfide, dimethyl sulfide, ethanol, isoprene, pentane, 1-decene, 1-heptene, 1-nonene, 1-octene, 3-methylhexane, (E)-2-nonene, ammonia, ethane, hydrogen sulfide, triethyl amine, and trimethyl amine.

7. The method of claim 1, wherein the analytic device is portable.

8. The method of claim 1, wherein a diagnosis of CDI indicates that the subject is at least 70% likely to have CDI.

9. The method of claim 1, wherein a diagnosis of CDI indicates that the subject is at least 80% likely to have CDI.

10. A method for treating a subject who has been diagnosed with Clostridioides difficile infection (CDI), wherein the method comprises:
obtaining a breath sample from the subject;
obtaining a VOC profile of the breath sample using an analytic device wherein the VOC profile comprises one or more of the VOCs detected and its corresponding quantity;
inputting one or more of the VOC quantities into a machine learning model stored in a non-transitory memory and implemented by a processor;
diagnosing the subject as having or not having CDI based on the output of the machine learning model;
and administering a treatment to a subject if the subject has been diagnosed with having CDI.

11. The method of claim 10, wherein the treatment comprises administration of metronidazole, vancomycin, fidaxomicin, or rifaximin.

12. The method of claim 10, wherein the treatment comprises fecal bacteriotherapy, probiotic therapy, or monoclonal antibody therapy.

13. The method of claim 10, wherein the machine learning model is developed using a population of patients with and without CDI.

14. The method of claim 10, wherein the analytic device is a selected-ion flow-tube mass spectrometry (SIFT-MS).

15. The method of claim 10, wherein the analytic device is a gas chromatograph.

16. The method of claim 10, wherein the one or more VOC quantities inputted into the machine learning model are selected from 2-propanol, acetaldehyde, acetone, acetonitrile, acrylonitrile, benzene, carbon disulfide, dimethyl sulfide, ethanol, isoprene, pentane, 1-decene, 1-heptene, 1-nonene, 1-octene, 3-methylhexane, (E)-2-nonene, ammonia, ethane, hydrogen sulfide, triethyl amine, and trimethyl amine.

17. The method of claim 10, wherein the VOC quantities inputted into the machine learning model comprise 2-propanol, acetaldehyde, acetone, acetonitrile, acrylonitrile, benzene, carbon disulfide, dimethyl sulfide, ethanol, isoprene, pentane, 1-decene, 1-heptene, 1-nonene, 1-octene, 3-methylhexane, (E)-2-nonene, ammonia, ethane, hydrogen sulfide, triethyl amine, and trimethyl amine.

18. The method of claim 10, wherein the analytic device is portable.

19. The method of claim 10, wherein a diagnosis of CDI indicates that the subject is at least 70% likely to have CDI.

20. The method of claim 10, wherein a diagnosis of CDI indicates that the subject is at least 80% likely to have CDI.
